(19) United States
(12) Patent Application Publication
(10) Pub. No.: US 2014/0343361 A1
(43) Pub. Date: Nov. 20, 2014

(54) Tip Protector for a Multi-Viewing Elements Endoscope

(71) Applicant: EndoChoice, Inc., Alpharetta, GA (US)

(72) Inventors: Golan Salman, Atlit (IL); Avi Levy, Herzliya (IL); Amram Aizenfeld, Ramot Menashe (IL)

(73) Assignee: EndoChoice, Inc., Alpharetta, GA (US)

(21) Appl. No.: 14/278,145

(22) Filed: May 15, 2014

Related U.S. Application Data
(60) Provisional application No. 61/824,228, filed on May 16, 2013.

(51) Int. Cl. A61B 1/00

(52) U.S. Cl. CPC A61B 1/00137
USPC 600/125; 600/121

(57) ABSTRACT

A tip protector for a multiple viewing element endoscope is provided, that protects the front as well as side viewing elements in the tip. In one embodiment, the protector comprises wings that act as shock absorbers. The protector is further designed such that the endoscope can washed without having to remove the protector from the tip.

### CROSS-REFERENCE TO RELATED APPLICATIONS

The present invention relies on U.S. Provisional Patent Application No. 61/824,228, entitled “Tip Protector For A Multi-Viewing Element Endoscope” and filed on May 16, 2013, for priority which is herein incorporated by reference in its entirety.

### FIELD

The present invention relates generally to an endoscope with multiple viewing elements, and more specifically, to an endoscope tip protector that protects the tip section of the endoscope and the components included within from damage.

### BACKGROUND

Endoscopes have attained great acceptance within the medical community, since they provide a means for performing procedures with minimal patient trauma, while enabling the physician to view the internal anatomy of the patient. Over the years, numerous endoscopes have been developed and categorized according to specific applications, such as cystoscopy, colonoscopy, laparoscopy, upper GI endoscopy among others. Endoscopes may be inserted into the body's natural orifices or through an incision in the skin.

An endoscope typically comprises an elongated tubular shaft, rigid or flexible, having a video camera or a fiber optic lens assembly at its distal end. The shaft is connected to a handle, which sometimes includes an ocular for direct viewing. Viewing is also usually possible via an external screen. Various surgical tools may be inserted through a working channel in the endoscope for performing different surgical procedures.

In most endoscopes, elements such as electronic components are packed together with fluid carrying elements in a small sized endoscope tip section. Further, the most important part of an endoscope, the camera is also located in the tip section. For example, U.S. patent application Ser. No. 13/119,032, by the Applicant of the present specification and incorporated herein by reference, describes the use of multiple cameras in a single endoscope for an improved field of view, which includes a front-pointing camera and multiple side-pointing cameras, which are located in the tip section.

Endoscopes are technologically sophisticated devices and are comprised of fragile components that are easily damaged or broken. Endoscope damage is often caused by damage to the tip section which might be due to a puncture at the distal and more flexible end of the endoscope or by dropping the tip section such that it hits the floor or another hard surface thereby damaging the cameras and other components located within the tip. It has been reported that over 60% of all endoscope repairs are done to address these types of damages and the average repair costs are quite high. The side components are especially vulnerable to damage during movement of the scope while it is not in use. Thus, there is a need in the art for a device that can protect endoscope tips from accidental damage.

### SUMMARY

A tip protector for a multiple viewing element endoscope is provided, that protects the front as well as side viewing elements in a tip section of an endoscope. In one embodiment, the tip protector comprises at least one shock absorber. The protector is further designed with minimum overlap with the tip surface, such that the endoscope can be washed without having to remove the protector from the tip section.

In one embodiment, the present specification describes a protective apparatus for protecting the components in a tip section of an endoscope from damage, comprising: a substantially cylindrical body, having a first end and a second end, wherein the tip section of the endoscope is inserted through the first end; at least two sets of windows placed along an axial length of the cylindrical body, wherein the axial length is measured from said first end to said second end; and, at least one shock absorbing structure positioned between adjacent at least two sets of windows.

In one embodiment, the protective apparatus further comprises a finger tab mechanism at the first end that is used to open or close the protective apparatus over the endoscope tip section so that the apparatus is secured on the tip section.

In an embodiment, the internal diameter of the substantially cylindrical body ranges from 3 mm to 20 mm.

In an embodiment, each of the at least two set of windows comprises three windows. Further, optionally, the windows are of equal dimensions. Still optionally, the windows are of different dimensions. In an embodiment, the windows have a length in the range of approximately 2 mm to 12 mm and a width in the range of approximately 3 mm to 13 mm.

In one embodiment, the protective apparatus further comprises at least two shock absorbing structures. Optionally, the shock absorbing structures are substantially rectangular. Optionally, the shock absorbing structures are rectangular in shape with rounded corners. In an embodiment, the shock absorbing structures have a length in the range of 20 mm to 40 mm and a width in the range of 2 mm to 12 mm. Optionally, each shock absorbing structure comprises at least two subsections of equal or different dimensions.

In another embodiment, the present specification is directed towards a protective apparatus for protecting components positioned in a tip section of an endoscope from damage, comprising: a substantially cylindrical body having a first portion comprising a first end and a second end and a second portion comprising a third end and a fourth end, wherein said first portion is, at its second end, structurally connected to said second portion at its third end using at least one connection point and wherein the protective apparatus can be opened or closed along the connection point; at least one set of windows placed along an axial length of the first portion, wherein said axial length is measured from the first end to the second end and wherein at least one set of windows is placed along an axial length of said second portion, wherein said axial length is measured from the third end to the fourth end; and, at least one shock absorbing structure positioned on each of said first portion and said second portion.

In one embodiment, said first portion and the second portion are substantially semi-cylindrical in shape. Further, the axial length of the first and second portions ranges from 30 mm to 40 mm.

In an embodiment, the first end of the first portion comprises a half-ring and the fourth end of the second portion comprises a half-ring that join together, when the protective apparatus is closed, to form a single collar for securing the tip protector on the tip section of an endoscope.

Optionally, the protective apparatus comprises at least one locking element for securing the apparatus in a closed position when placed on the endoscope tip section.

When in a closed position, the internal diameter of the substantially cylindrical tip protector ranges from 3 mm to 20 mm.

In an embodiment, each set of windows comprises two windows. The windows have a length in the range of approximately 2 mm to 12 mm and a width in the range of approximately 3 mm to 13 mm.

In an embodiment, the protective apparatus further comprises three shock absorbing structures on each of the first and second portions positioned between adjacent sets of windows. In an embodiment, when the apparatus is in a closed position, at least one of the shock absorbing structures on each of the first and second portions clasp together to form a single structure. Optionally, when said first and second portions are in a closed position, the total number of shock absorbing structures is four. Optionally, the shock absorbing structures are rectangular in shape. Optionally, the shock absorbing structures have a length in the range of 20 mm to 40 mm and a width in the range of 2 mm to 12 mm.

In yet another embodiment, the present specification describes a protective apparatus for protecting the components in the tip section of an endoscope from damage, comprising: a substantially cylindrical body, having a first end, a second end, an inner surface, and an outer surface, wherein the tip section of the endoscope can be inserted therethrough at the first end; at least two sets of windows placed along an axial length of the outer surface of the substantially cylindrical body, wherein said axial length is measured from said first end to said second end; at least one shock absorbing structure positioned between adjacent at least two sets of windows; and, at least one set of flanges and associated grooves positioned along a circumference of the inner surface of the substantially cylindrical body.

Optionally, the at least one set of flanges and associated grooves aid in inserting and securing the tip section of the endoscope inside the protective apparatus.

Optionally, the flanges are made of an elastic material that bend backward when the tip section of endoscope is inserted inside the apparatus and retract back to secure the tip section in its place once the tip section is inserted.

Optionally, two sets of flanges and associated grooves are positioned along the circumference of the inner surface of the substantially cylindrical body wherein a first set is positioned at a first end within which the endoscope tip is inserted and a second set is positioned near a middle portion of inner surface of the apparatus.

Optionally, the flanges in said first set and said second set are of equal size.

Optionally, the size of the flanges in said first set is larger than the size of the flanges in said second set.

Optionally, the size of the flanges in said first set is smaller than the size of the flanges in said second set.

In an embodiment, the internal diameter of the substantially cylindrical body ranges from 3 mm to 20 mm.

In yet another embodiment, the present specification is directed towards a protective device for protecting a tip of an endoscope from damage, wherein the endoscope has a distal face with a first optical element and a first discrete illuminator and curved sides with a second optical element and a second discrete illuminator, comprising: a cylindrical body having a distal face, curved sides, and a proximal end, wherein the proximal end is open to receive said tip, wherein the distal face comprises a first void to expose said first optical element and first discrete illuminator, and wherein the curved sides comprises a plurality of second voids placed along an axial length of the cylindrical body; and a plurality of shock absorbing structures positioned along the axial length of the cylindrical body and extending outward from said cylindrical body.

In an embodiment, the cylindrical body comprises four substantially equal-sized quadrants defined by the intersection of a first plane that extends along the axial length of the cylindrical body and a second plane that extends along the axial length of the cylindrical body, wherein said second plane is perpendicular to the first plane and wherein each quadrant comprises at least one of said plurality of second voids to expose said second optical element and said second discrete illuminator and wherein each quadrant comprises at least one of said plurality of shock absorbing structures.

In an embodiment, each quadrant comprises at least two of said plurality of second voids and wherein said at least two voids are positioned serially along the axial length of the cylindrical body. Optionally, the at least two voids have equal surface areas and have equal peripheries. In an embodiment, each of the at least two voids have a length in the range of approximately 2 mm to 12 mm and a width in the range of approximately 3 mm to 13 mm. Optionally, the at least two voids are separated by a portion of the cylindrical body having a width in a range 1 mm to 3 mm.

Optionally, the at least one of said plurality of shock absorbing structures is rectangular in shape. In an embodiment, the at least one of said plurality of shock absorbing structures has a length in the range of 20 mm to 40 mm and a width in the range of 2 mm to 12 mm.

Optionally, the first void is circular, has a diameter that is less than the diameter of the distal face, and is positioned substantially in a center of the distal face.

In yet another embodiment, the present specification described a protective device for protecting a tip of an endoscope from damage, wherein the endoscope has a distal face with a first optical element and a first discrete illuminator, a first curved side with a second optical element and a second discrete illuminator, and a second curved side with a third optical element and a third discrete illuminator, comprising: a cylindrical body having a distal face, curved sides, and a proximal end, wherein the proximal end is open to receive said tip and wherein the distal face comprises a first void to expose said first optical element and first discrete illuminator; and a plurality of shock absorbing structures positioned along the axial length of the cylindrical body and extending outward from said cylindrical body, wherein the cylindrical body comprises four substantially equal-sized quadrants defined by the intersection of a first plane that extends along the axial length of the cylindrical body and a second plane that extends along the axial length of the cylindrical body, wherein said second plane is perpendicular to the first plane and wherein at least two of said quadrants comprise a plurality of second voids one of said plurality of shock absorbing structures.

In an embodiment, each of said at least two quadrants comprise at least two of said plurality of second voids and wherein said at least two voids are positioned serially along the axial length of the cylindrical body.

In an embodiment, in a first of the at least two quadrants, the at least two of said plurality of second voids are positioned to expose the second optical element and the second discrete illuminator. In an embodiment, in a second of the at least two quadrants, the at least two of said plurality of second voids are positioned to expose the third optical element and the third discrete illuminator. Optionally, the at least two voids have equal surface areas and have equal peripheries. Still optionally, each of the at least two voids has a length in the range of 2 mm to 12 mm and a width in the range of 3 mm to 13 mm. Optionally, the at least two voids are separated by a portion of the cylindrical body having a width in a range of 1 mm to 3 mm.

Optionally, the at least one of the plurality of shock absorbing structures is rectangular in shape. Optionally, the at least one of the plurality of shock absorbing structures has a length in the range of 20 mm to 40 mm and a width in the range of 2 mm to 12 mm.

The aforementioned and other embodiments of the present shall be described in greater depth in the drawings and detailed description provided below.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 illustrates a cross section of a multiple viewing elements endoscope, according to one embodiment;

FIG. 2 shows a cross-sectional view of a tip section of a multiple viewing elements endoscope, according to some embodiments;

FIG. 3A shows one embodiment of a tip protector with a finger tab in a closed position;

FIG. 3B shows one embodiment of the tip protector with the finger tab shown in FIG. 3A in an open position;

FIG. 4 illustrates another embodiment of a tip protector in an open position;

FIG. 5 illustrates another embodiment of a tip protector in a closed position;

FIG. 6 illustrates the comparative diameters of various sections of an endoscope that are used in configuring the appropriate dimensions of the tip protector;

FIG. 7 illustrates another embodiment of the tip protector of the present specification;

FIG. 8A illustrates an embodiment of the tip protector described in FIG. 7 with the endoscope tip inserted inside the tip protector;

FIG. 8B illustrates a side view of the tip protector described in FIG. 7 with the endoscope tip inserted inside the tip protector in accordance with an embodiment of the present specification;

FIG. 9 illustrates a view of the proximal end of the tip protector along with the endoscope tip inserted inside the tip protector in accordance with an embodiment of the present specification;

FIG. 10 illustrates a cross-sectional view of the tip protector in accordance with an embodiment of the present specification;

FIG. 11 illustrates a cross-sectional view of the tip protector in accordance with another embodiment of the present specification; and,

FIG. 12 illustrates a cross-sectional view of the tip protector in accordance with another embodiment of the present specification.

### DETAILED DESCRIPTION

In one embodiment, the present specification describes a protective apparatus that protects the tip section of an endoscope and the components included within from accidental damage. In some embodiments, the tip protector also acts as a shock absorber to prevent damage during a fall. Further, the tip protector is designed such that it is easy to attach and remove. In one embodiment, the tip protector is adapted to be cleaned with the scope and/or when attached to the scope.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In one embodiment, the tip section of the endoscope comprises multiple viewing elements. In one embodiment, the multiple viewing elements typically comprise a lens assembly such as a camera and/or a fiber optic lens assembly along with image sensors, to deliver a multi-part display. According to another embodiment, at least one of the multiple viewing elements is positioned at a distal (front) end surface of the tip section and points forward, and at least one of the remaining multiple viewing elements are positioned further back in the tip section, and point to the sides of the endoscope. According to another embodiment, all of the multiple viewing elements are positioned in proximity to or at the distal end of the tip section and point sideways.

The multiple viewing elements and optionally other elements that exist in the tip section (such as a light source, a working channel, and/or a fluid injector) are uniquely scaled, configured and packaged so that they fit within the constrained space available inside the tip section, without compromising on output quality. The tip protector is designed to protect the components in all the above mentioned embodiments and configurations.

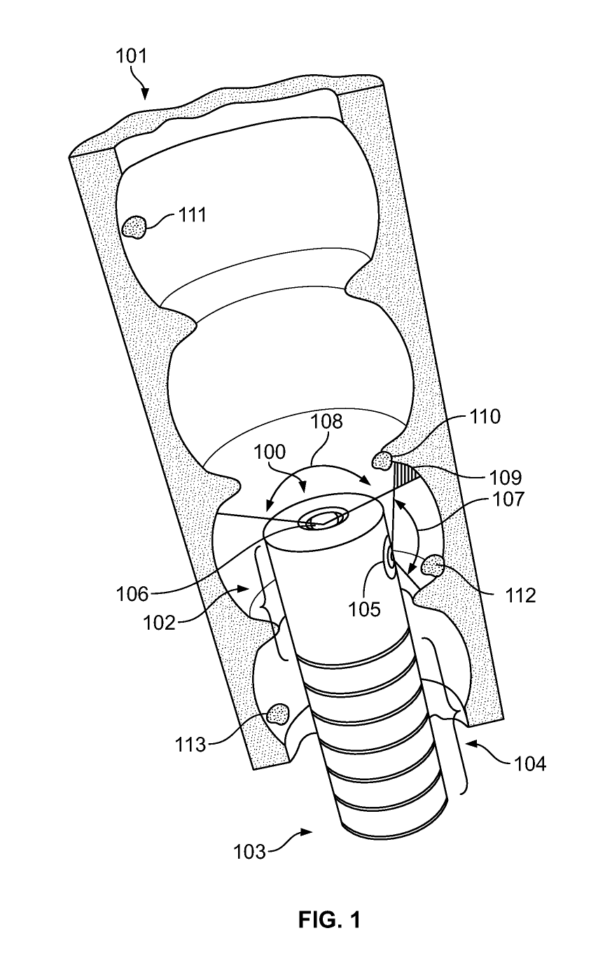

Reference is now made to FIG. 1 which is a cross-sectional view of a multiple viewing element endoscope 100 inserted inside a portion or lumen 101 of a human body, according to an embodiment. Endoscope 100 includes an elongated shaft 103 (a portion of which is shown) terminating with a tip section 102, which is turnable by way of a bending section 104. In an embodiment, tip section 102 includes a front-pointing viewing element 106 as well as at least one side-pointing viewing element 105. While front-pointing viewing element 106 can detect, based on its field of view 108, polyps such as polyps 110 and 111, at least one side-pointing viewing element 105 is further able to detect, based on its field of view 107, polyps which are normally hidden from the front-pointing viewing element, such as polyp 112. In an embodiment, by rotating endoscope 100 around its longitudinal axis, at least one side-pointing viewing element 105 can detect polyps circumferentially, for a field of view that is greater than 300 degrees around the endoscope. This enables the detection of polyps such as a polyp 113, which is, similar to polyp 112, located on an inner side of a fold. Area 109 represents the overlapping section between the two fields of view 107 and 108. Any abnormality in the area 109 can be detected by both side viewing element 105 and the front viewing element 106. In other configurations (not shown) of the present specification, two or more side-pointing viewing elements are placed in the tip section, each having a different field of view.

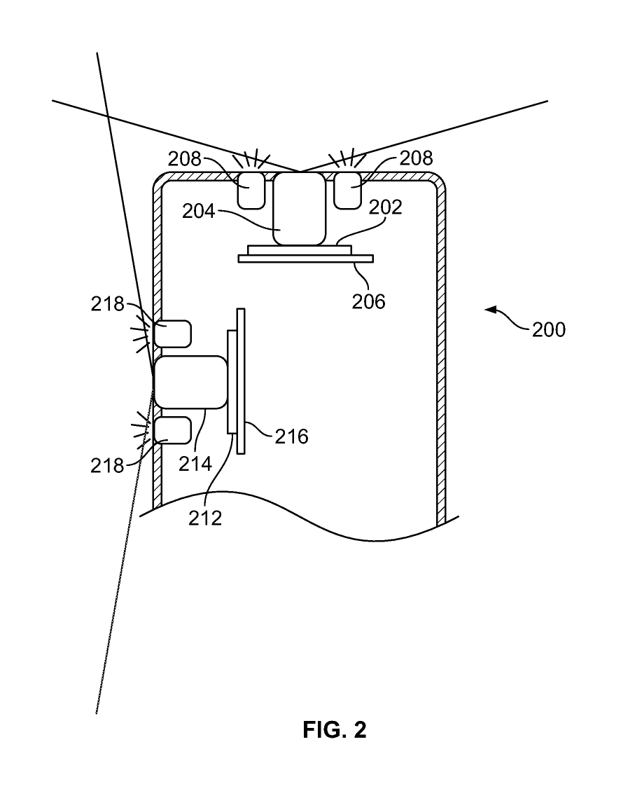

Reference is now made to FIG. 2, which illustrates a cross-sectional view of a tip section 200 of a multiple viewing element endoscope, according to an embodiment. In an embodiment, tip section 200 includes a front-pointing image sensor 202, such as but not limited to Charge Coupled Device (CCD) or a Complementary Metal Oxide Semiconductor (CMOS) image sensor. In an embodiment, front-pointing image sensor 202 is mounted on a circuit board 206, which supplies front-pointing image sensor 202 with necessary electrical power, and in an embodiment, generates still images and/or video feeds captured by the image sensor. In one embodiment, the circuit board 206 is connected to a set of electrical cables (not shown) which are threaded through a channel running through the elongated shaft of the endoscope. In an embodiment, the front-pointing image sensor 202 has a lens assembly 204 mounted on top of it and provides the necessary optics for receiving images. In one configuration, the lens assembly 204 includes a plurality of lenses, static or movable, which provides a field of view of at least 90 degrees and up to essentially 180 degrees.

In an embodiment, one or more discrete front illuminators 208 are placed next to lens assembly 204, for illuminating its field of view. In one embodiment, the discrete front illuminators 208 are attached to the same circuit board 206 upon which the front-pointing image sensor 202 is mounted.

In an embodiment of the present specification, the tip section 200 further includes a side-pointing image sensor 212, such as but not limited to Charge Coupled Device (CCD) or a Complementary Metal Oxide Semiconductor (CMOS) image sensor, mounted on the circuit board 216. In one embodiment, the side-pointing image sensor 212 has a lens assembly 214 mounted on top of it and provides the necessary optics for receiving images. In an embodiment, one or more discrete side illuminators 218 are placed next to lens assembly 214, for illuminating its field of view. In one embodiment, the discrete side illuminators 218 are attached to the same circuit board 216 on which side-pointing image sensor 212 is mounted.

In another configuration, the circuit boards described above comprise a single integrated circuit board on which both the front and the side-pointing image sensors are mounted. In one embodiment, optionally, there are two side-pointing image sensors. Optionally, side-pointing image sensors and their respective lens assemblies are advantageously positioned relatively close to the distal end surface of the tip section 200, shown in FIG. 2. For example, in one embodiment, the center of the side-pointing viewing element (which is the center axis of the side-pointing image sensor and associated lens assembly) is positioned approximately 7 to 11 millimeters from the distal end of the tip section. This is enabled by an advantageous miniaturizing of the front and side-pointing multiple viewing elements, which allows for enough internal space in the tip section for angular positioning of the cameras without colliding.

It would be apparent to one of ordinary skill in the art that the side-pointing viewing elements or cameras, and their associated optics and electronics are particularly susceptible to damage in case the endoscope falls or its tip hits a hard surface and/or during cleaning process of the endoscope. Thus, the present specification provides a tip protector that not only covers the front of the endoscope tip, but also the side portions of the endoscope tip.

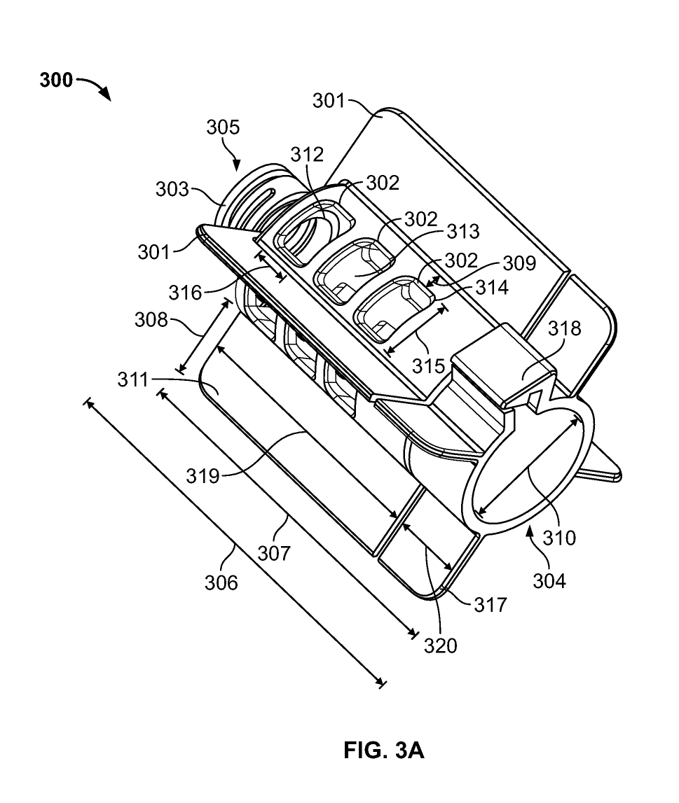

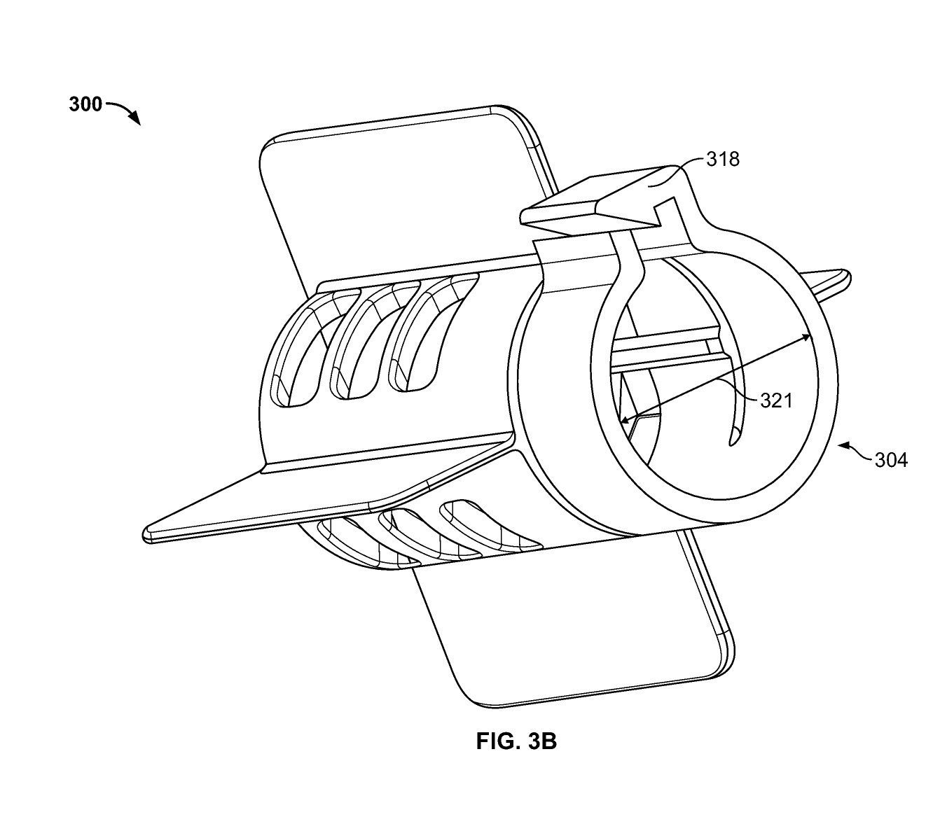

FIG. 3A illustrates a tip protector 300, according to one embodiment of the present specification. In an embodiment, the tip protector 300 has a first end or proximal end 304 and a second end or distal end 305. In an embodiment, the endoscope tip is inserted in the tip protector 300 through the first end or proximal end 304 of the tip protector 300. In some embodiments, when inserted, elements of the endoscope tip are positioned such that they align with corresponding openings, windows or other protective elements of tip protector 300, as described in greater detail below.

As shown in FIG. 3A, the tip protector 300 is in a closed position. Tip protector 300 is designed in a substantially cylindrical shape, such that it covers the endoscope tip section on all sides. In one embodiment, the endoscope tip protector 300 comprises a substantially cylindrical body having an axial length 306, measured from first end 304 to second end 305, ranging from 30 mm to 40 mm and an internal diameter having a range of 5 mm to 20 mm.

In one embodiment, the tip protector is designed for a minimum overlap with the tip surface, so that it does not interfere with general maintenance of the endoscope. In one embodiment, the tip protector is adapted to be cleaned with the scope, when attached to the scope. The tip protector, in one embodiment, comprises a plurality of windows 302 on its external surface along the axial length. Windows 302 allow for washing of the endoscope without having to remove the protector 300. In one embodiment, the tip protector is designed such that it can be put into a washing machine while still attached to the endoscope tip. Since windows 302 ensure a minimum overlap with the tip surface, the cleaning or washing quality is not affected.

In one embodiment, the tip protector 300 comprises at least two sets of windows 302. In one embodiment, the tip protector comprises a plurality of sets of windows 302 ranging in number from two to twelve, placed around the cylindrical body of the tip protector. In one embodiment, each set of windows 302 comprises three window areas or voids, of substantially equal area, arranged serially along the axial length 306 of the tip protector. A first void 312 begins approximately 0.5 mm to 2 mm from a distal end 305 of the tip protector, depending upon the length of threaded portion 303, and extends approximately 7 mm along the axial length of the tip protector as represented by length 316 in FIG. 3A. A second void 313 is separated from the first void 312 by a distance ranging from 1 mm to 3 mm. In one embodiment, the second void 313 has the same axial length and width as the first window 312. A third void 314 is separated from the second void 313 by a distance ranging from 1 mm to 3 mm. In one embodiment, the third void 314 has the same axial length and width as the first void 312 and the second voids 313. In one embodiment, the width 315 of the windows 302 is approximately 8 mm.

It may be appreciated that the windows along the axial length are necessary to expose the side components of the endoscopic tip for cleaning. Concurrently however, a mechanism is necessitated to protect the components exposed by the windows from damage. In one embodiment, the mechanism includes flanges, extending members, wings, or other shock absorbing structures. Therefore in one embodiment, each set of windows is separated by a shock absorbing structure.

Referring back to FIG. 3, in one embodiment, the tip protector 300 is equipped with at least two wings 301 placed around the circumference of the tip protector, such that each of the wings is positioned between two adjacent sets of windows and runs along at least a portion of axial length 306 of tip protector 300. In one embodiment, the tip protector 300 is equipped with two to nine wings 301 positioned around the circumference. When the tip protector is placed on the endoscope tip, wings 301, in one embodiment, act as shock absorbers in case of an accidental fall or if the endoscope tip hits a hard surface, thus protecting the multiple viewing elements. In one embodiment, each wing has a substantially rectangular structure. In one embodiment, at least one of the corners of each wing are rounded. Further, in an embodiment, each wing is of the same size, with a length 307 in the range of 20 mm to 40 mm and a width 308 in the range of 2 mm to 12 mm. In one embodiment, each wing is placed at a distance of 2 mm to 4 mm on either side of and between adjacent sets of windows as represented by the distance 309 in FIG. 3A. In one embodiment, each wing has two subsections 311 and 317, wherein subsection 311 is longer than subsection 317. In one embodiment, subsection 311 has a length 319 in the range of 15 mm to 30 mm and subsection 317 has a length 320 in the range of 5 mm to 10 mm. The smaller subsections 317 are located towards the proximal end 304 of the tip protector and nearer to the bending section (component 104 of FIG. 1) of the endoscope's tip to further protect the tip in the area wherein the bending section may be located when the tip protector is put on the endoscope tip. Division of wings into multiple subsections enhances their overall shock absorbance quality. In one embodiment, each wing has more than two subsections of the same or different dimensions.

In one embodiment, an element 303, which is shown protruding from the distal end 305 of the tip protector 300 is positioned such that it helps in protecting the front portion of the tip section of the endoscope from damage. In an embodiment, the element 303 comprises a threaded surface which helps avoid accumulation of debris internally.

In one embodiment, a finger tab 318 is provided on the proximal end 304 of the tip protector which helps the user to maneuver the tip protector onto the tip section or during removal of the tip section. The physician can open the finger tab 318 while placing the tip protector over the tip section of the endoscope and can close the finger tab once the tip protector is in place over the tip section. When the tip protector 300 is in a closed position, the internal diameter 310 of a substantially circular front face of the proximal end 304 of the tip protector 300 is such that the tip section can be securely positioned into the tip protector. In one embodiment, the internal diameter 310 ranges from 3 mm to 20 mm.

In one embodiment, the wings are also used to snap the tip protector open or shut to enable attachment or removal of the tip protector from the endoscope tip.

It may be appreciated that the number of windows, their sizes and the difference between individual windows may be different in different configurations of the tip protector, so long as it serves the purpose of allowing adequate protection for the tip section and/or adequate cleaning of the tip without removing the protector.

Similarly, the wings or flanges may have a different size, shape or position than that described in the embodiment above, so long as they provide adequate shock absorption and prevent damage to the components within the tip.

FIG. 3B illustrates the tip protector described in FIG. 3A, with the finger tab 318 shown in an open position as per an embodiment of the present specification. There are multiple mechanisms through which the finger tab system is operated. In one embodiment, the user can push down the finger tab to open or close it. While the finger tab 318 is in open position, the diameter 321 of the circular front face of proximal end 304 of the tip protector 300 is slightly higher than the diameter 310 illustrated in FIG. 3A, when the finger tab is in a closed position. Thus, when the tip protection 300 is in an open position, internal diameter 321 is larger than internal diameter 310. The increase in diameter allows for easier placement of the tip protector over the tip section of the scope. Subsequently, as the finger tab 318 is closed, the reduced internal diameter 310 enables the tip protector 300 to remain secured in its position and not slide in any direction.

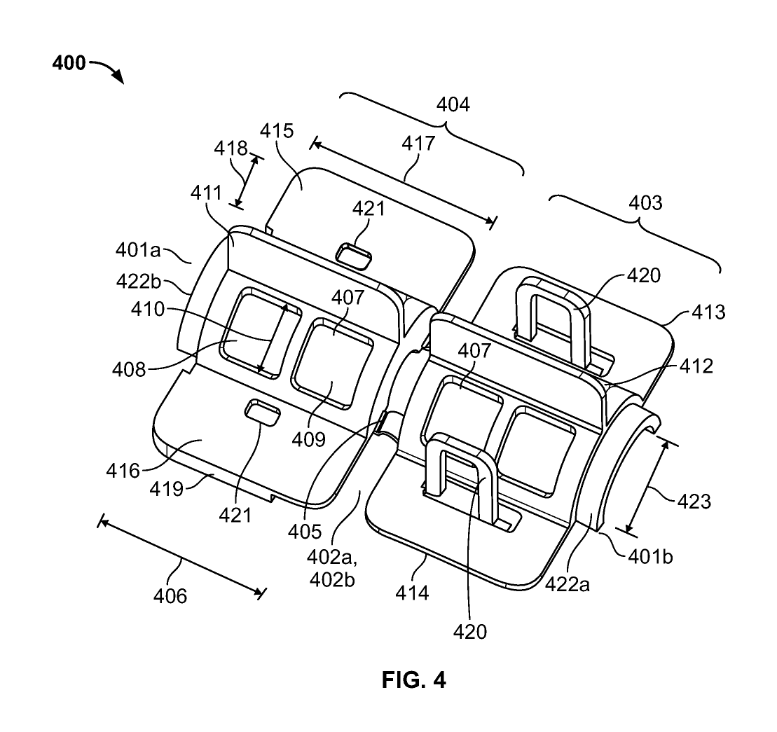

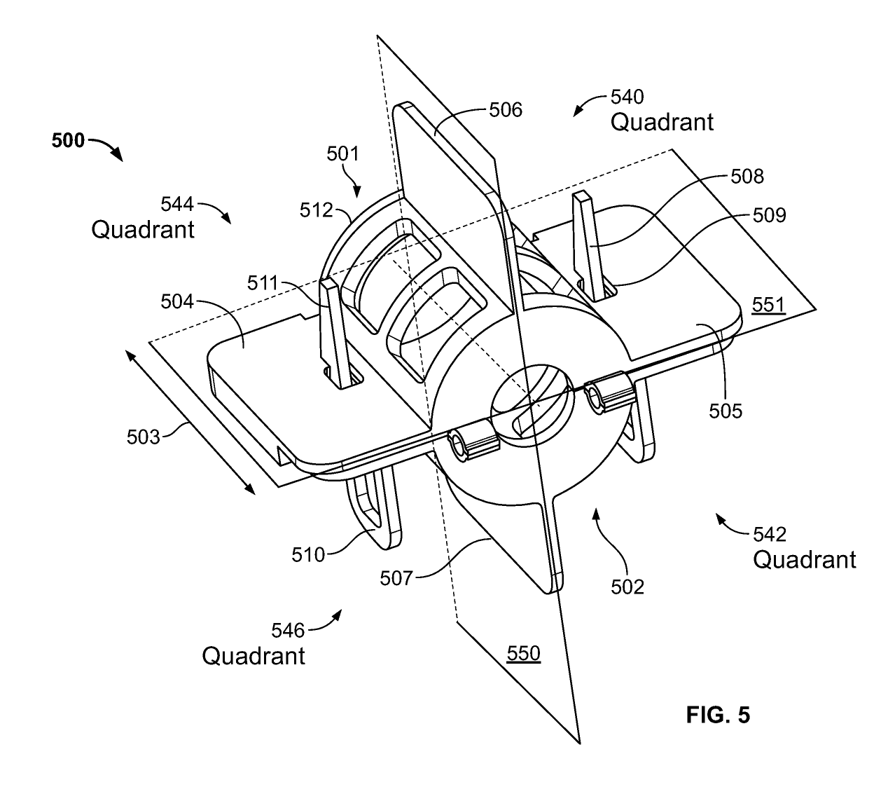

FIGS. 4 and 5 illustrate another configuration for the tip protector in accordance with an embodiment of the present specification. As shown in FIG. 4, in an embodiment, the tip protector comprises a first portion 403 and a second portion 404. First portion 403 has a first end 401a and a second end 402a. Second portion 404 has a third end 402b and a fourth end 401b. First portion 403 is structurally connected at its second end 402a to second portion 404 at its third end 402b, using at least one connection point 405. As shown in FIG. 4, second end 402a and third end 402b form a middle portion between the first and second portions, when the tip protector is in an open position. When folded about the second end 402a and third end 402b, the tip protector is in a closed position, as shown in FIG. 5.

Referring back to FIG. 4, tip protector 400, in one embodiment, has a substantially cylindrical shape that covers the endoscope tip section from all sides when closed. In one embodiment, first portion 403 and second portion 404 of the tip protector each have a substantially semi-cylindrical shape with an axial length 406 ranging from 30 mm to 40 mm. When closed, tip protector 400 has an internal diameter 423 in the range of 5 mm to 20 mm.

To enable a minimum overlap with the tip surface, and to facilitate washing of the endoscope tip with the protector on, the tip protector in one embodiment, comprises a plurality of sets of windows 407 on its external surface along the axial length 406. In one embodiment, the tip protector comprises four sets of windows 407 such that two sets are positioned on first portion 403 and two sets are placed on second portion 404. In one embodiment, each set of windows 407 comprises two windows or voids 408 and 409 of substantially equal area, arranged serially along the axial length 406 of the cylindrical tip protector. The first void 408 begins approximately 2 mm to 5 mm from the proximal end 401 of the tip and extends 2 mm to 12 mm, and preferably 5 mm to 10 mm along the axial length 406. First void 408 has a width 410 ranging from 3 mm to 13 mm, and preferably 5 mm to 10 mm. The second 409 void is separated from the first void 408 by a distance ranging from 2 mm to 5 mm. In one embodiment, the second void 409 has the same axial length and width 408 as the first void.

In one embodiment, each set of windows 407 is separated by a shock absorbing structure, to protect the components exposed by the windows from damage. In one embodiment, the tip protector 400 is equipped with six wings, with three wings placed on each of the first portion 403 and the second portion 404. In each of the portions 403 and 404, wings 412 and 411 are placed, respectively, between two adjacent sets of windows 407. Wings 413 and 414 on first portion 403 and winds 415 and 416 on second portion 404 are positioned on either side of the semi-cylindrical body of the respective portions. The wings act as shock absorbers in case of an accidental fall or if the endoscope tip hits a hard surface and serve to protect the multiple viewing elements. In one embodiment, each wing has a substantially rectangular structure. Further, each wing is of the same size, with a length 417 ranging from 20 mm to 40 mm a width 418 in the range of 2 mm to 12 mm. In one embodiment, each wing is placed at a distance of 2 mm to 5 mm from its neighboring sets of windows on either side.

In one embodiment, the wings are equipped with clasps 419 that enable wings on opposite sides to clasp together when the tip protector is closed at the middle.

In one embodiment, the tip protector is equipped with locking elements 420 on the first portion 403. Corresponding grooves 421 are provided on the second portion 404, which allows the tip protector to be locked when in closed position, and prevents opening during normal movement. In one embodiment, locking elements are located on the side wings on the first portion 403 while corresponding grooves are located in the side wings of the second portion 404. It should be noted herein that the locking elements and corresponding grooves may be positioned on either the first portion, second portion, or a combination thereof, as long as it achieves the objectives of the present invention.

In one embodiment, tip protector 400 includes a first half ring 422a on the first portion 403 and a second half ring 422b on the second portion 404. When the tip protector 400 is closed over the endoscope tip second the two half rings 422a and 422b join to form a collar, which provides a structure for securing the tip protector in place over the tip section.

FIG. 5 illustrates the tip protector of FIG. 4 in a closed position in accordance with an embodiment of the present specification. Referring to FIG. 5, the tip protector 500 comprises a proximal end 501 and a distal end 502. In an embodiment, the cylindrical body has an axial length 503 ranging from 30 mm to 40 mm and an internal diameter in the range of 5 mm to 20 mm. In this position, the two wings on the either side of the two portions 403 and 404 (as described with reference to 413,414,415, and 416 in FIG. 4) clasp together with corresponding wings on the opposite side, to form a single wing units 504 and 505. In one embodiment, the thickness of the combined wings 504 and 505 is in range of 2 mm to 4 mm, while the thickness of the individual wings 506 and 507 is in the range of 1 mm to 2 mm.

In an embodiment, in a closed position the tip protector 500 comprises a substantially cylindrical body that is formed from four quadrants of equal size. The four quadrants 540, 542, 544, and 546 are defined by the intersection of a first plane 550 that extends along the axial length of the cylindrical body and a second plane 551 that is perpendicular to the first plane but also extends along the axial length of the cylindrical body. In one embodiment, first plane 550 is in the same plane as wings 506 and 507. In one embodiment, second plane 551 is in the same plane as wings 504 and 505. In some embodiments, first plane 550 is defined by wings 506 and 507 and second plane 551 is defined by wings 504 and 505.

When the tip protector 500 is closed, the locking elements 508 fit into corresponding grooves 509 to lock the tip protector in a closed position. In one embodiment, locking elements 508 are pressed so that they can be released from grooves 509, thereby opening the tip protector. In one embodiment, a first end 510 of each locking element is broad, in the range of 10 mm to 13 mm, to allow the user to conveniently maneuver the locking mechanism. A second end 511 of the locking element, which fits into the grooves, is slender and its thickness is approximately 9 mm. The grooves 509 have a width of approximately 4 mm. The locking mechanism does not interfere with washing and general maintenance of the endoscope tip as it is located on the side wings and away from the main cylindrical body of the tip protector. It would be appreciated that any other type of locking or gripping mechanism may be used for the endoscope tip protector. Collar 512, which is formed by the combination of two half rings 422a and 422b, shown in FIG. 4, provides adequate grip for holding the tip protector in its position.

One of ordinary skill in the art would appreciate that while the tip protector is designed such that it is simple to put on and remove from the tip section of the endoscope, the tip protector is further provided with a grip so that it does not fall off during regular movement of the scope. Further, the grip of the tip protector does not affect washing quality when the endoscope tip section is cleaned.

In one embodiment, the tip protector is made from plastic materials. In another embodiment, the tip protector is made from sponge materials. In another embodiment, the tip protector is fabricated to be re-usable or disposable.

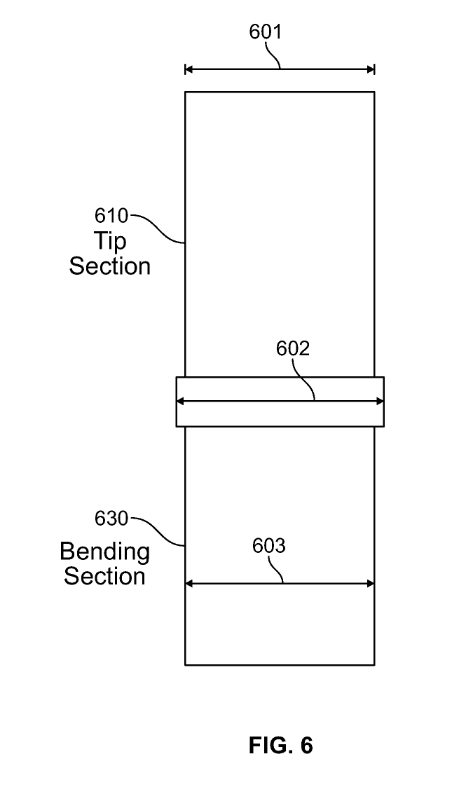

FIG. 6 illustrates the comparative diameters of various sections of an endoscope that are used in configuring the appropriate dimensions of the tip protector. Tip section 610, in an embodiment, has a diameter 601 while the intermediate area between the tip section 610 and bending section 630 has a diameter 602 (the bending section has been described with respect to FIG. 1). Referring back to FIG. 6, the bending section 30 has a diameter 603. Typically, the proportion between the three diameters along the endoscope is: 603≦601≦602. In one embodiment, diameter 601 is approximately in the range of 7 mm to 13 mm.

Thus, the tip protector is designed such that it effectively holds the endoscope at these three different points, in spite of varying diameters. Also, the internal dimensions or diameters of the tip protector are configured such that it grips both the bending section 630 and tip section 610, without hindering the cleaning and washing of these parts.

Referring back to FIG. 3B and FIG. 6 simultaneously, in one embodiment, finger tab 318 of tip protector 300, typically has a diameter 321 in an open position when introduced to endoscope tip. Diameter 321 is typically larger than diameter 602, shown in FIG. 6, which is the diameter of the intermediate area between the tip and bending section. As tip protector 300 is placed on the endoscope tip section, the finger tab 318 is closed around the intermediate area between the tip and bending sections such that the tip protector has a smaller diameter 310 (as illustrated in FIG. 3A). Typically diameter 310 is smaller than 602 the diameter of the intermediate area between the tip and bending sections. Thus, when is use, the tip protector is held around the endoscope tip without falling off the tip.

Referring to FIGS. 4 and 6, simultaneously, in one embodiment, tip protector 400 includes a half ring 422a on the first portion 403 and a half ring 422b on the second portion 404, as described earlier. When protector 400 is closed over endoscope tip, the two half rings 422a and 422b form a single collar having a diameter smaller than 602, which is the diameter of the intermediate area between the tip and bending sections. This ensures that the tip protector retains its grip despite the varying diameters of bending section, intermediate section and the tip section.

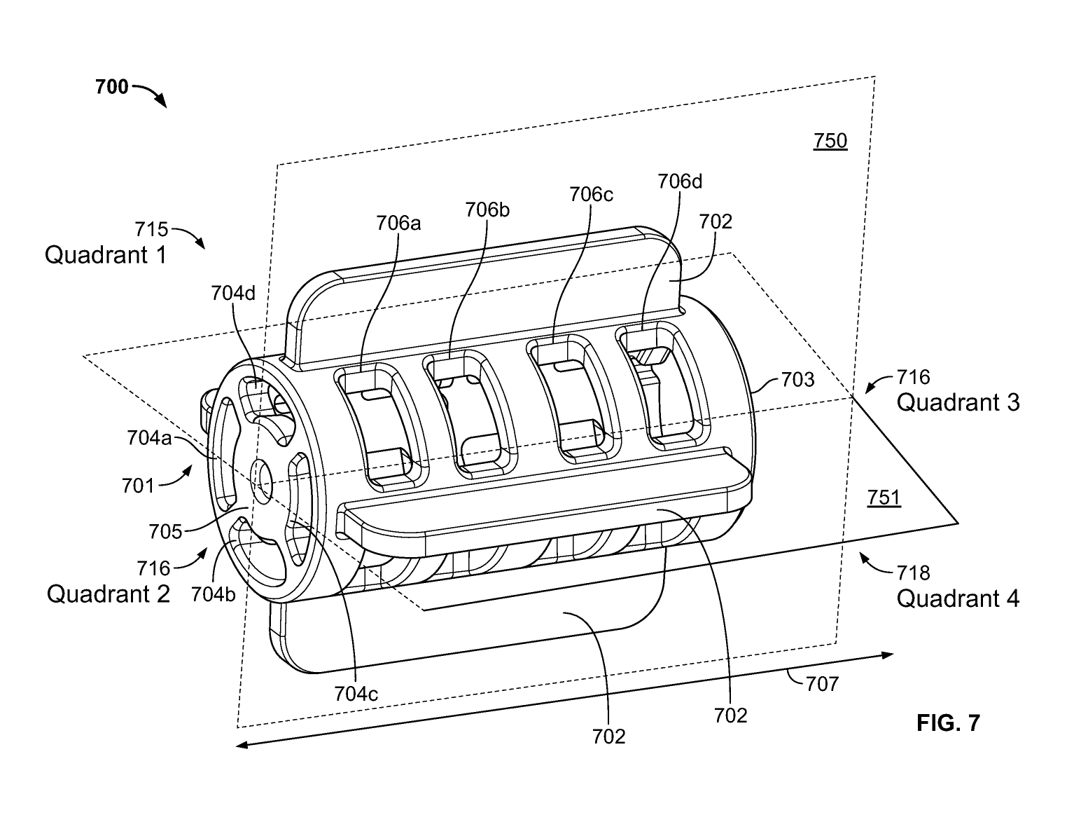

FIG. 7 illustrates another configuration of the tip protector in accordance with an embodiment of the present specification. In an embodiment, the tip protector 700 is a single component that slides over the tip section of the scope and remains there until the physician/nurse/user removes it. The tip protector 700 is configured such that it protects the fragile camera lenses and the illuminators windows from scratches and breakage during scope transport, reprocessing and storage. Further, the design allows the reprocessing fluids to contact the surfaces of the tip section of the scope. It also allows for appropriate airflow to reach the distal tip section of the scope to facilitate dry time and minimize growth of microorganisms while the scope is in storage.

The tip protector 700 comprises a substantially cylindrical body with a first end or distal end 701 and a second end or proximal end 703 and curved sides that form the body. To place the tip protector 700 over the endoscope, the tip section of an endoscope is inserted into the proximal end 703 of the tip protector 700 and is gently pushed until the entire tip portion fits into the tip protector 700. In an embodiment, the tip protector 700 is equipped with a fitting mechanism such that when pushed inside, the endoscope fits into the tip protector and remains secured until the physician/nurse/user removes it. In an embodiment, to remove the tip protector 700, the physician can pull the endoscope tip out of the tip protector 700 with minimal effort to release it.

Referring to FIG. 7, a first window or void 705 is positioned at the center of an external face of the first end or distal end 701 of the tip protector 700 that also allows for the washing of various components located on the endoscope tip section while still affording protection for the components. In one embodiment, first window or void 705 corresponds to at least a first optical element and a first discrete illuminator that are positioned on a distal face or front surface of the tip section of an endoscope, as described above. The design of the first end or distal end of the tip protector 700 enables protection of the components, such as at the least one viewing element (first optical element and first discrete illuminator) located on the distal front surface of the endoscope tip section against any accidental shock or damage. In one embodiment, window 705 is circular and has a diameter that is less than the diameter of the first end or distal end 701 of the tip protector 700.

In an optional embodiment, the tip protector 700 comprises a plurality of windows or voids 704a, 704b, 704c and 704d positioned on an external face of the first end or distal end 701 that allow for washing of various components located on the front surface of the tip section of endoscope without having to remove the tip protector 700. In an embodiment, the windows or voids 704a, 704b, 704c and 704d are uniform in shape and size. In an embodiment, the voids have equal surface areas and equal peripheries. In an alternate embodiment, the shape and size of each of the windows 704a, 704b, 704c and 704d are configured to provide adequate space between the windows and corresponding components located on the tip section of the endoscope during the washing operation.

In an embodiment, the tip protector 700 comprises a plurality of second windows or voids 706a, 706b, 706c, and 706d positioned around the curved external cylindrical side surfaces along its axial length 707 that allows for washing of the endoscope tip section, without having to remove the tip protector 700. In an embodiment, the tip protector 700 comprises multiple sets of second windows or voids located along its circumference. In an embodiment, the length of the second windows or voids ranges from 2 mm to 12 mm. In an embodiment, the width of the second windows or voids ranges from 3 mm to 13 mm. In an embodiment, the distance between adjacent voids ranges from 1 mm to 3 mm. In an embodiment, adjacent voids are separated by a portion of the cylindrical body.

It may be appreciated that the windows along the axial length are necessary to expose the side components of the endoscopic tip for cleaning. In some embodiments, at least two of the plurality of second windows are positioned on curved sides of the tip protector and correspond to at least a second optical element and a second discrete illuminator. In some embodiments, at least two of the plurality of second windows or voids is positioned on curved sides of the tip protector and correspond to a third optical element and a third discrete illuminator.

In addition, a mechanism is necessitated to protect the components exposed by the windows from damage. Therefore, in an embodiment, the tip protector 700 comprises a plurality of shock absorbing structures 702, such as wings, that protect the internal components of the tip section of endoscope against any accidental fall or shock. In an embodiment, the plurality of shock absorbing structures or wings is substantially rectangular in shape. In an embodiment, the plurality of shock absorbing structures has a length ranging from 20 mm to 40 mm and a width in the range of 2 mm to 12 mm.

In an embodiment, tip protector 700 comprises a substantially cylindrical body that is formed from four quadrants of substantially equal size. The four quadrants 715, 716, 717, 718 are defined by the intersection of a first plane 750 that extends along the axial length of the cylindrical body and a second plane 751 that is perpendicular to the first plane 750 but also extends along the axial length of the cylindrical body. In one embodiment, each quadrant 715, 716, 717, and 718 comprises at least a portion of at least one shock absorbing structure.

In an embodiment, at least two the quadrants comprise at least two of the plurality of second voids or windows 702 which are positioned along the axial length 707 of the cylindrical body. In an embodiment, in a first of the at least two quadrants, the at least two of the plurality of second voids or windows 702 are positioned to expose the second optical element and the second discrete illuminator of the endoscopic tip section described above. In an embodiment, in a second of the at least two quadrants, the at least two of the plurality of second voids or windows 702 are positioned to expose the third optical element and the third discrete illuminator or the endoscopic tip section described above.

In an embodiment, the tip protector 700 is a semi-rigid device and is developed in either reusable or low cost disposable versions. In an embodiment, the tip protector 700 is designed such that a single size fits all versions of endoscope sizes. In one alternate embodiment, the tip protector 700 is customized for scopes of different sizes.

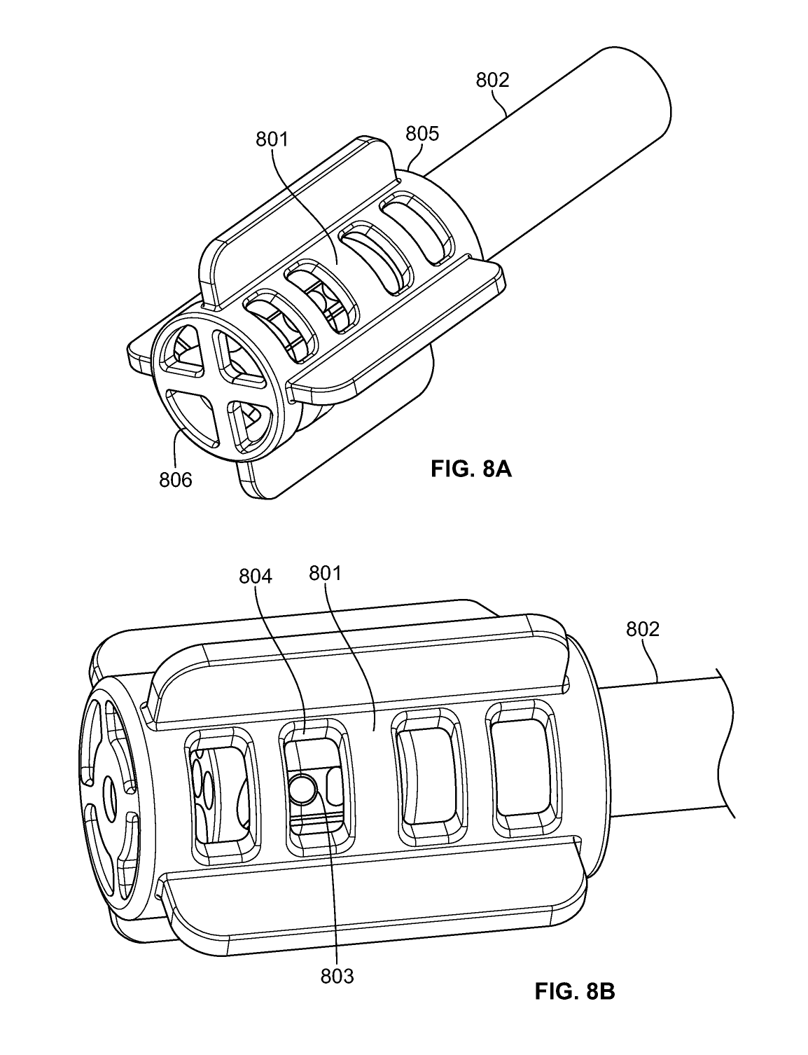

FIGS. 8A and 8B illustrate two different views of the tip protector described in FIG. 7 with the endoscope tip section inserted inside the tip protector in accordance with an embodiment of the present specification. Referring to FIG. 8A, the tip protector 801, having a second end or proximal end 805 and a first end or distal end 806, is shown placed over the endoscope tip section. The tip section of the insertion tube 802 of the endoscope is shown covered by the tip protector 801. In an embodiment, the tip protector 801 is a single component equipped with a fitting mechanism. When the insertion tube 802 is slightly pushed inside the tip protector 801 through its proximal end 805, it settles into its position and remains secured until the physician/nurse/user removes it by pulling out the insertion tube 802. In FIG. 8B, side viewing element 803 is shown such that it can be accessed through the window 804 during washing operation. The plurality of windows such as 804, located on the surface of the tip protector, are configured such that various components located on the endoscope tip section can be easily accessed during washing operation without having to remove the tip protector 801.

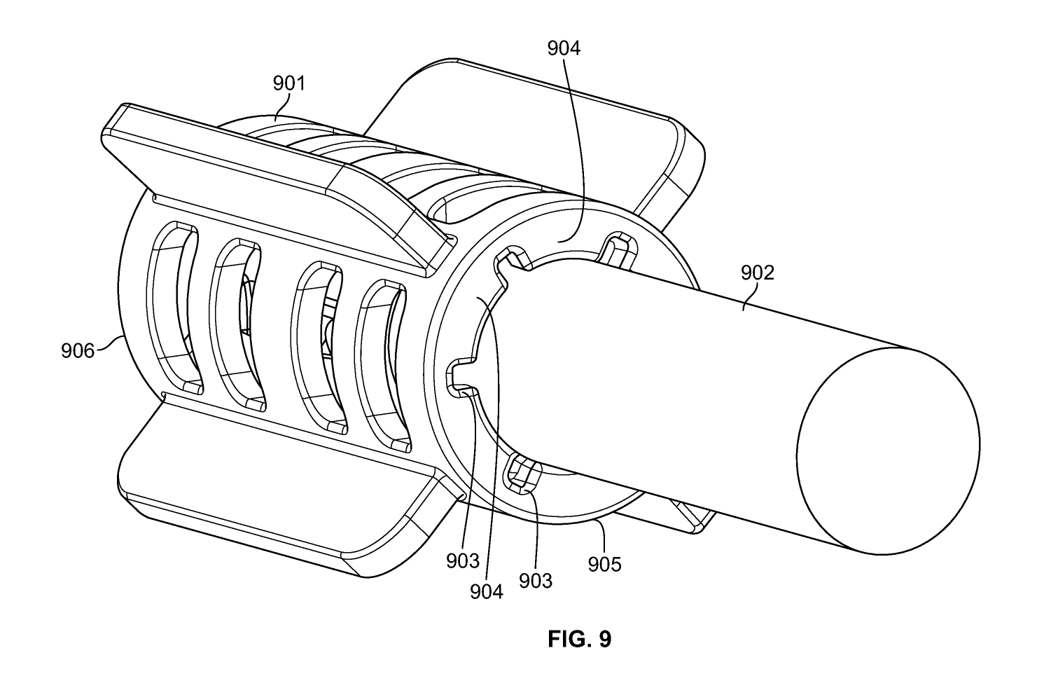

FIG. 9 illustrates rear view of the tip protector described in FIG. 7 with the endoscope tip section inserted inside the tip protector in an embodiment of the present specification. As shown in FIG. 9, tip protector 901 comprises a proximal end 905 and distal end 906 and is placed over the tip section of insertion tube 902 of the endoscope. The proximal end 905 of the tip protector 901 comprises a plurality of grooves 903 such that these grooves aid in placing or removing the tip protector over the endoscope tip section with minimal effort. In an embodiment the grooves 903 are rectangular in shape and are positioned at equal distances along the circumference of the outer edge of the proximal end 905 of the tip protector 900. A plurality of flanges 904 is located between the grooves 903. The flanges 904 are designed such that when the endoscope is pushed inside the tip protector 901 through its proximal end 905, flanges 904 bend backwards and allow for the endoscope tip section to slide inside the tip protector 901. Subsequently, as the tip protector 901 slides over the complete tip section, the flanges 904 further aid in keeping the endoscope secured inside the tip protector 901 as they retract back to the original position to enclose the tip section. In an embodiment, the flanges 904 are made from elastic materials such as rubber or other fibers.

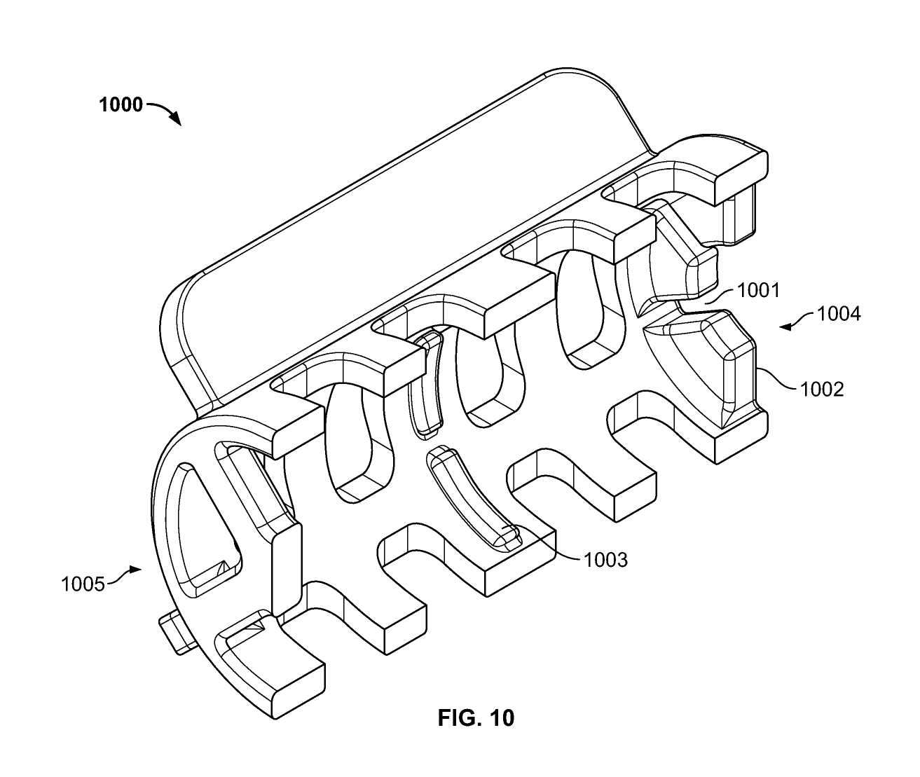

FIG. 10 illustrates a cross-sectional view of the tip protector described in FIG. 7, in accordance with an embodiment of the present specification. As shown in FIG. 10, the tip protector 1000 comprises a proximal end 1004 and a distal end 1005 with a plurality of grooves 1001 located on its proximal end 1004 that enables placing and removing the tip protector 1000 on the endoscope tip with minimal effort. In one embodiment, a plurality of flanges 1002 are positioned between adjacent grooves 1001 at the proximal end 1004 of tip protector 1000. The grooves 1001 and associated flanges 1002 enable the placement of endoscope tip inside the tip protector. In an embodiment, the tip protector 1000 further comprises a second set of flanges 1003 which are placed on the inside portion of the tip protector 1000. The second set of flanges 1003 further helps to keep the endoscope tip secured within the tip protector 1000. In an embodiment, the first set of flanges 1002 are longer than the second set of flanges 1003. The flanges 1002 and 1003 are designed such that when the endoscope is pushed into the tip protector 1000, flanges 1002 and 1003 may bend backward and allow for the endoscope to slide inside the tip protector 1000. Subsequently, as the tip protector 1000 slides over the complete tip section, the flanges 1002 and 1003 help in keeping the endoscope secured inside the tip protector 1000 as they retract back to the original position to enclose the tip section. In an embodiment, the flanges 1002 and 1003 are made from elastic materials such as rubber or other fiber with elasticity.

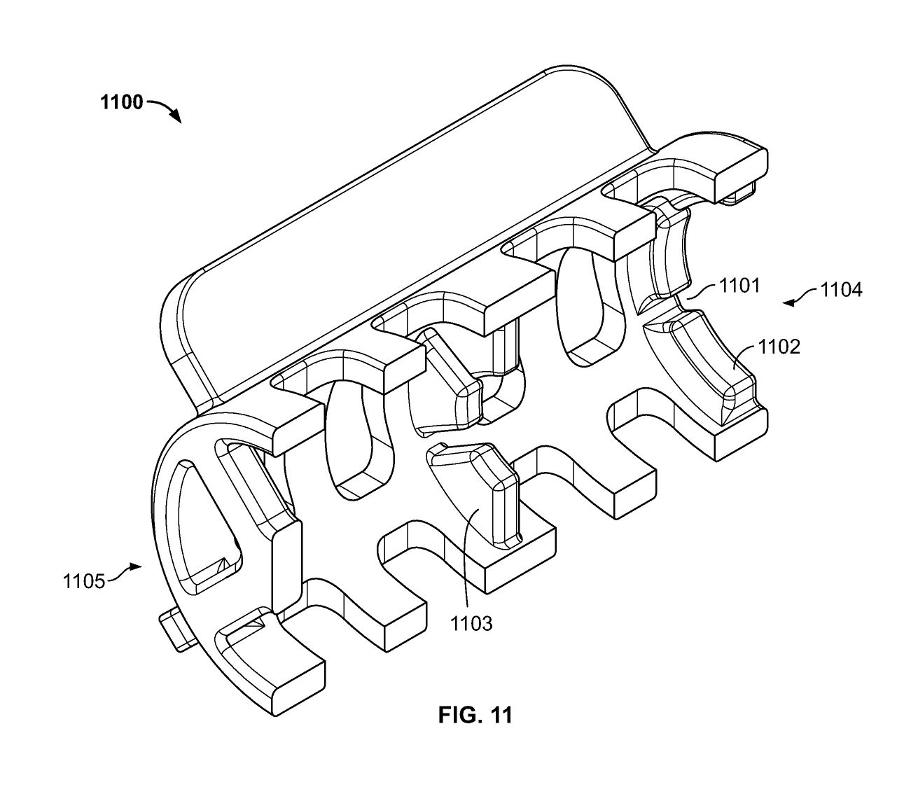

FIG. 11 illustrates a cross-sectional view of the tip protector described in FIG. 7, in accordance with another embodiment of the present specification. As shown in FIG. 11, the tip protector 1100 comprises a proximal end 1104 and a distal end 1105 with a plurality of grooves 1101 located on its proximal end 1104. A plurality of short flanges 1102 are placed between adjacent grooves 1101. The tip protector further comprises a second set of flanges 1103 which are placed on the inside portion of the tip protector 1100. In an embodiment, the first set of flanges 1102 is shorter than the second set of flanges 1103.

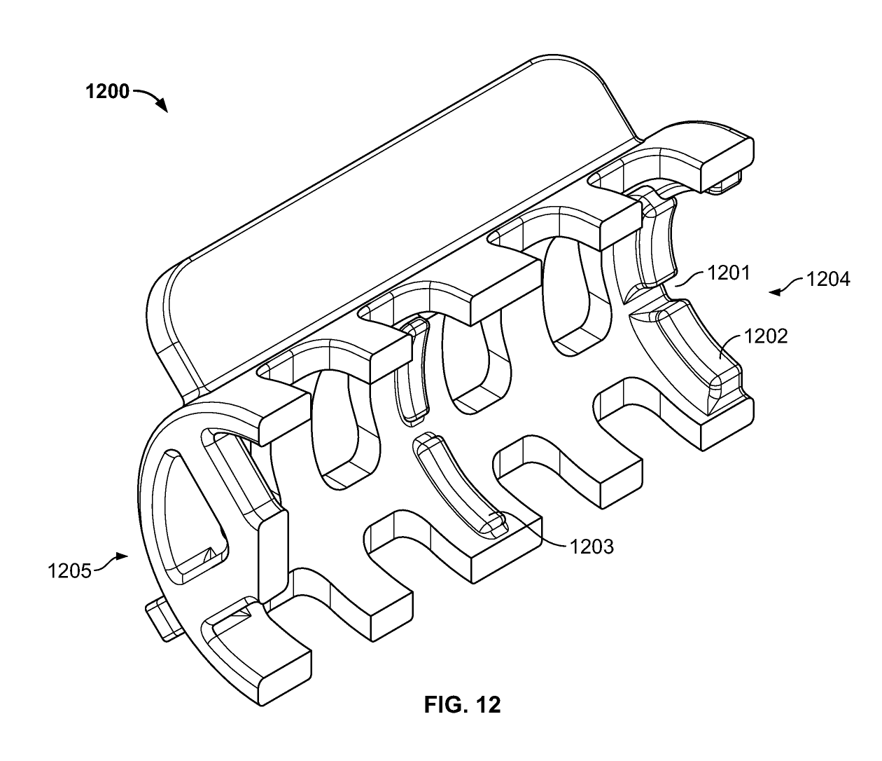

FIG. 12 illustrates a cross-sectional view of the tip protector described in FIG. 7, in accordance with another embodiment of the present specification. As shown in FIG. 12, the tip protector 1200 comprises a proximal end 1204 and a distal end 1205 with a plurality of grooves 1201 located on its proximal end 1204. A plurality of flanges 1202 are placed between adjacent grooves 1201. The tip protector further comprises a second set of flanges 1203 which are placed on the inside portion of the tip protector 1200. In this embodiment, the first set of flanges 1202 is equal in length to the second set of flanges 1203.

It may be appreciated that various size and shapes of flanges can be used to secure the endoscope tip inside the tip protector 1200 without departing from the spirit and scope of this specification While the above embodiment show only two sets of flanges, one may appreciate that that multiple sets of flanges can be used to practice this invention.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A protective apparatus for protecting the components in a tip section of an endoscope from damage, comprising:
a substantially cylindrical body, having a first end and a second end, wherein the tip section of the endoscope is inserted through the first end;
at least two sets of windows placed along an axial length of the cylindrical body, wherein the axial length is measured from said first end to said second end; and,
at least one shock absorbing structure positioned between adjacent at least two sets of windows.

2. The protective apparatus of claim 1 further comprising a finger tab mechanism at the first end that is used to open or close the protective apparatus over the endoscope tip section so that the apparatus is secured on the tip section.

3. The protective apparatus of claim 1 wherein the internal diameter of the substantially cylindrical body ranges from 3 mm to 20 mm.

4. The protective apparatus of claim 1 wherein each of the at least two set of windows comprise three windows.

5. The protective apparatus of claim 1 wherein the windows are of equal dimensions.

6. The protective apparatus of claim 1 wherein the windows are of different dimensions.

7. The protective apparatus of claim 1 wherein the windows have a length in the range of approximately 2 mm to 12 mm and a width in the range of approximately 3 mm to 13 mm.

8. The protective apparatus of claim 1 further comprising at least two shock absorbing structures.

9. The protective apparatus of claim 1 wherein the shock absorbing structures are substantially rectangular.

10. The protective apparatus of claim 9 wherein the shock absorbing structures have a length in the range of 20 mm to 40 mm and a width in the range of 2 mm to 12 mm.

11. The protective apparatus of claim 1 wherein the shock absorbing structures are rectangular in shape with rounded corners.

12. The protective apparatus of claim 1 wherein each shock absorbing structure comprises at least two subsections of equal or different dimensions.

13. A protective apparatus for protecting components positioned in a tip section of an endoscope from damage, comprising:
a substantially cylindrical body having a first portion comprising a first end and a second end and a second portion comprising a third end and a fourth end, wherein said first portion is, at its second end, structurally connected to said second portion at its third end using at least one connection point and wherein the protective apparatus can be opened or closed along the connection point;
at least one set of windows placed along an axial length of the first portion, wherein said axial length is measured from the first end to the second end and wherein at least one set of windows is placed along an axial length of said second portion, wherein said axial length is measured from the third end to the fourth end; and,
at least one shock absorbing structure positioned on each of said first portion and said second portion.

14. The protective apparatus of claim 13, wherein said first portion and the second portion are substantially semi-cylindrical in shape.

15. The protective apparatus of claim 13 wherein the axial length of the first and second portions ranges from 30 mm to 40 mm.

16. The protective apparatus of claim 13 wherein the first end of the first portion comprises a half-ring and the fourth end of the second portion comprises a half-ring that join together, when the protective apparatus is closed, to form a single collar for securing the tip protector on the tip section of an endoscope.

17. The protective apparatus of claim 13 further comprising at least one locking element for securing the apparatus in a closed position when placed on the endoscope tip section.

18. The protective apparatus of claim 13 wherein an internal diameter of the substantially cylindrical tip protector, when in a closed position, ranges from 3 mm to 20 mm.

19. The protective apparatus of claim 13 wherein each set of windows comprises two windows.

20. The protective apparatus of claim 19 wherein the windows have a length in the range of approximately 2 mm to 12 mm and a width in the range of approximately 3 mm to 13 mm.

21. The protective apparatus of claim 13 further comprising three shock absorbing structures on each of the first and second portions positioned between adjacent sets of windows.

22. The protective apparatus of claim 21, wherein when the apparatus is in a closed position, at least one of the shock absorbing structures on each of the first and second portions clasp together to form a single structure.

23. The protective apparatus of claim 22, wherein when the apparatus is in a closed position, a total number of shock absorbing structures is four.

24. The protective apparatus of claim 21 wherein the shock absorbing structures are rectangular in shape.

25. The protective apparatus of claim 24 wherein the shock absorbing structures have a length in the range of 20 mm to 40 mm and a width in the range of 2 mm to 12 mm.

26. A protective apparatus for protecting the components in the tip section of an endoscope from damage, comprising:
a substantially cylindrical body, having a first end, a second end, an inner surface, and an outer surface, wherein the tip section of the endoscope can be inserted therethrough at the first end;
at least two sets of windows placed along an axial length of the outer surface of the substantially cylindrical body, wherein said axial length is measured from said first end to said second end;
at least one shock absorbing structure positioned between adjacent at least two sets of windows; and,
at least one set of flanges and associated grooves positioned along a circumference of the inner surface of the substantially cylindrical body, for aiding in inserting and securing the tip section of the endoscope inside the protective apparatus.

27. The protective apparatus of claim 26 wherein said flanges are made of an elastic material that bend backward when the tip section of endoscope is inserted inside the apparatus and retract back to secure the tip section in its place once the tip section is inserted.

28. The protective apparatus of claim 26 comprising two sets of flanges and associated grooves positioned along the circumference of the inner surface of the substantially cylindrical body wherein a first set is positioned at a first end within which the endoscope tip is inserted and a second set is positioned near a middle portion of the apparatus.

29. The protective apparatus of claim 28 wherein the flanges in said first set and said second set are of equal size.

30. The protective apparatus of claim 28 wherein the size of the flanges in said first set is larger than the size of the flanges in said second set.

31. The protective apparatus of claim 28 wherein the size of the flanges in said first set is smaller than the size of the flanges in said second set.

32. The protective apparatus of claim 26 wherein the internal diameter of the substantially cylindrical body ranges from 3 mm to 20 mm.

33. A protective device for protecting a tip of an endoscope from damage, wherein the endoscope has a distal face with a first optical element and a first discrete illuminator and curved sides with a second optical element and a second discrete illuminator, comprising:
a cylindrical body having a distal face, curved sides, and a proximal end, wherein the proximal end is open to receive said tip, wherein the distal face comprises a first void to expose said first optical element and first discrete illuminator, and wherein the curved sides comprises a plurality of second voids placed along an axial length of the cylindrical body; and
a plurality of shock absorbing structures positioned along the axial length of the cylindrical body and extending outward from said cylindrical body.

34. The protective device of claim 33 wherein the cylindrical body comprises four substantially equal-sized quadrants defined by the intersection of a first plane that extends along the axial length of the cylindrical body and a second plane that extends along the axial length of the cylindrical body, wherein said second plane is perpendicular to the first plane and wherein each quadrant comprises at least one of said plurality of second voids to expose said second optical element and said second discrete illuminator and wherein each quadrant comprises at least one of said plurality of shock absorbing structures.

35. The protective device of claim 34 wherein each quadrant comprises at least two of said plurality of second voids and wherein said at least two voids are positioned serially along the axial length of the cylindrical body.

36. The protective device of claim 35 wherein the at least two voids have equal surface areas and have equal peripheries.

37. The protective device of claim 35 wherein each of the at least two voids have a length in the range of approximately 2 mm to 12 mm and a width in the range of approximately 3 mm to 13 mm.

38. The protective device of claim 35 wherein the at least two voids are separated by a portion of the cylindrical body having a width in a range 1 mm to 3 mm.

39. The protective device of claim 33 wherein the at least one of said plurality of shock absorbing structures is rectangular in shape.

40. The protective device of claim 39 wherein the at least one of said plurality of shock absorbing structures has a length in the range of 20 mm to 40 mm and a width in the range of 2 mm to 12 mm.

41. The protective device of claim 33 wherein the first void is circular, has a diameter that is less than the diameter of the distal face, and is positioned substantially in a center of the distal face.

42. A protective device for protecting a tip of an endoscope from damage, wherein the endoscope has a distal face with a first optical element and a first discrete illuminator, a first curved side with a second optical element and a second discrete illuminator, and a second curved side with a third optical element and a third discrete illuminator, comprising:
a cylindrical body having a distal face, curved sides, and a proximal end, wherein the proximal end is open to receive said tip and wherein the distal face comprises a first void to expose said first optical element and first discrete illuminator; and
a plurality of shock absorbing structures positioned along the axial length of the cylindrical body and extending outward from said cylindrical body, wherein the cylindrical body comprises four substantially equal-sized quadrants defined by the intersection of a first plane that extends along the axial length of the cylindrical body and a second plane that extends along the axial length of the cylindrical body, wherein said second plane is perpendicular to the first plane and wherein at least two of said quadrants comprise a plurality of second voids one of said plurality of shock absorbing structures.

43. The protective device of claim 42 wherein each of said at least two quadrants comprise at least two of said plurality of second voids and wherein said at least two voids are positioned serially along the axial length of the cylindrical body.

44. The protective device of claim 43 wherein in a first of the at least two quadrants, the at least two of said plurality of second voids are positioned to expose the second optical element and the second discrete illuminator.

45. The protective device of claim 44 wherein in a second of the at least two quadrants, the at least two of said plurality of second voids are positioned to expose the third optical element and the third discrete illuminator.

46. The protective device of claim 43 wherein the at least two voids have equal surface areas and have equal peripheries.

47. The protective device of claim 43 wherein each of the at least two voids has a length in the range of 2 mm to 12 mm and a width in the range of 3 mm to 13 mm.

48. The protective device of claim 43 wherein the at least two voids are separated by a portion of the cylindrical body having a width in a range of 1 mm to 3 mm.

49. The protective device of claim 42 wherein at least one of said plurality of shock absorbing structures is rectangular in shape.

50. The protective device of claim 42 wherein at least one of said plurality of shock absorbing structures has a length in the range of 20 mm to 40 mm and a width in the range of 2 mm to 12 mm.
